(19) United States
(12) Patent Application Publication
(10) Pub. No.: US 2023/0123814 A1
(43) Pub. Date: Apr. 20, 2023

(54) USE OF ALR1 GENE OR ALR1 PROTEIN OF ALUMINUM ION RECEPTOR IN REGULATING PLANT ALUMINUM RESISTANCE

(71) Applicants: Zhejiang University, Hangzhou (CN); Guangdong Laboratory for Lingnan Modern Agriculture, Guangzhou City (CN)

(72) Inventors: Shaojian ZHENG, Hangzhou (CN); Zhongjie DING, Hangzhou (CN); Chen XU, Hangzhou (CN); Guixin LI, Hangzhou (CN); Jingying YAN, Hangzhou (CN)

(21) Appl. No.: 17/797,264

(22) PCT Filed: May 20, 2021
(86) PCT No.: PCT/CN2021/094884

§ 371 Date: Aug. 3, 2022

(30) Foreign Application Priority Data
Apr. 6, 2021 (CN) 202110367334.2

(51) Int. Cl. C12N 15/82; C07K 14/415

(52) U.S. Cl. CPC C12N 15/8271; C07K 14/415

(57) ABSTRACT

The present disclosure belongs to the technical field of plant genetic engineering, and in particular relates to use of an ALR1 gene or an ALR1 protein of an aluminum ion receptor in regulating plant aluminum resistance. In the present disclosure, a primers includes an upstream primer with a nucleotide sequence shown in SEQ ID NO: 1 and a downstream primer with a nucleotide sequence shown in SEQ ID NO: 2. The primer is capable of improving the plant aluminum resistance and promoting growth of a plant root system, thereby promoting absorption of nutrients and water by the plant, which is of great significance for improving a yield of crops.

### CROSS REFERENCE TO RELATED APPLICATION

The present application claims priority to the Chinese Patent Application No. 202110367334.2, filed with the China National Intellectual Property Administration (CNIPA) on Tuesday, Apr. 6, 2021, and entitled “USE OF ALR1 GENE OR ALR1 PROTEIN OF ALUMINUM ION RECEPTOR IN REGULATING PLANT ALUMINUM RESISTANCE”, which is incorporated herein by reference in its entirety.

### TECHNICAL FIELD

The present disclosure belongs to the technical field of plant genetic engineering, and in particular relates to use of an ALR1 gene or an ALR1 protein of an aluminum ion receptor in regulating plant aluminum resistance.

### BACKGROUND ART

Soil acidification and subsequent aluminum toxicity are widespread problems in agricultural production worldwide. The intensification of soil acidification has greatly affected the yield of crops, posing a great threat to sustainable agricultural production. The decrease in soil pH leads to the leaching of base ions, and causes some insoluble mineral aluminum (the most abundant metal element in the crust, with an average content of about 8%) to dissociate into ionic state and enter soil solution. A large number of studies have shown that a micromolar level of aluminum ions may significantly inhibit the growth of plant root systems, thereby affecting the absorption of nutrients and water by the root systems, which ultimately leads to the reduction of crop yields, or even to the end of production. Therefore, aluminum toxicity is recognized as a major limiting factor for acid soil affecting crop production. Although the activity of aluminum in soil can be traditionally reduced through soil improvement methods (such as applying lime and other amendments), these measures require a lot of manpower, material resources and financial resources on the one hand, and are still very difficult to transform the soil at a solid substructure on the other hand. It is an effective way to continuously and efficiently solve aluminum toxicity in acid soil by obtaining plant varieties with strong resistance to aluminum toxicity through genetic improvement.

The physiological mechanisms of plant aluminum resistance can be divided into an aluminum exclusion mechanism represented by changes in the properties of organic acids and cell wall components, and an aluminum internal tolerance mechanism represented by aluminum storage in vacuoles for compartmentalization. These two mechanisms are currently recognized as the most important physiological mechanisms for plants to resist aluminum toxicity. With the rapid development of molecular biology, a large number of genes and proteins have also been identified to involve in a signal transduction pathway of the plant aluminum resistance. However, nothing is known about how plants sense aluminum ions. It is of great significance for further clarifying a molecular mechanism of plant aluminum resistance, providing reference for breeding, and improving the plant aluminum resistance and crop yield by identifying genes involved in sensing aluminum ions and deeply analyzing their functions. The aluminum ion receptor gene and its function that regulate the plant aluminum resistance have not yet been discovered, which hinders the cultivation of aluminum-resistant plants to a certain extent.

### SUMMARY

An objective of the present disclosure is to provide use of an ALR1 gene or an ALR1 protein of an aluminum ion receptor in regulating plant aluminum resistance. A primer provided by the present disclosure is capable of improving the plant aluminum resistance and promoting growth of a plant root system, thereby promoting absorption of nutrients and water by the plant and improving a yield of crops.

The present disclosure provides a primer of an ALR1 gene regulating plant aluminum resistance, where the primer includes an upstream primer with a nucleotide sequence shown in SEQ ID NO: 1 and a downstream primer with a nucleotide sequence shown in SEQ ID NO: 2.

The present disclosure further provides a vector for overexpressing an ALR1 gene of an Arabidopsis thaliana aluminum ion receptor prepared based on the primer, where the vector uses 35s-pCAMBIA1301 as a vector backbone, and further includes an ALR1 gene of the Arabidopsis thaliana aluminum ion receptor; and the ALR1 gene has a nucleotide sequence shown in SEQ ID NO: 3.

The present disclosure further provides use of an ALR1 gene or an ALR1 protein of an Arabidopsis thaliana aluminum ion receptor or the primer in regulating plant aluminum resistance, where the ALR1 gene has a nucleotide sequence shown in SEQ ID NO: 3, and the ALR1 protein has an amino acid sequence shown in SEQ ID NO: 4.

The present disclosure further provides use of an ALR1 gene or an ALR1 protein for overexpressing an Arabidopsis thaliana aluminum ion receptor or the vector in improving plant aluminum resistance, where the ALR1 gene has a nucleotide sequence shown in SEQ ID NO: 3, and the ALR1 protein has an amino acid sequence shown in SEQ ID NO: 4.

The present disclosure further provides use of an ALR1 gene or an ALR1 protein of an Arabidopsis thaliana aluminum ion receptor or the primer in regulating a root system elongation of an aluminum-stressed plant, where the ALR1 gene has a nucleotide sequence shown in SEQ ID NO: 3, and the ALR1 protein has an amino acid sequence shown in SEQ ID NO: 4.

The present disclosure further provides use of an ALR1 gene or an ALR1 protein for overexpressing an Arabidopsis thaliana aluminum ion receptor or the vector in regulating a root system elongation of an aluminum-stressed plant, where the ALR1 gene has a nucleotide sequence shown in SEQ ID NO: 3, and the ALR1 protein has an amino acid sequence shown in SEQ ID NO: 4.

Preferably, the root system may be a primary root.

The present disclosure further provides use of an ALR1 gene or an ALR1 protein of an Arabidopsis thaliana aluminum ion receptor or the primer in regulating a root system aluminum content of an aluminum-stressed plant, where the ALR1 gene has a nucleotide sequence shown in SEQ ID NO: 3, and the ALR1 protein has an amino acid sequence shown in SEQ ID NO: 4.

The present disclosure further provides use of an ALR1 gene or an ALR1 protein for overexpressing an Arabidopsis thaliana aluminum ion receptor or the vector in regulating a root system aluminum content of an aluminum-stressed plant, where the ALR1 gene has a nucleotide sequence shown in SEQ ID NO: 3, and the ALR1 protein has an amino acid sequence shown in SEQ ID NO: 4.

Preferably, the plant may include Arabidopsis thaliana.

The present disclosure provides a primer of an ALR1 gene regulating plant aluminum resistance. In the present disclosure, the ALR1 gene of the aluminum ion receptor is cloned from a model plant Arabidopsis thaliana by specific amplification primers, and two mutant lines are constructed by knocking out and overexpressing the ALR1 gene; aluminum resistance is significantly reduced in a knockout line, while an overexpression line shows a significant increase in the aluminum resistance. The test results show that under A1 stress, overexpression transgenic Arabidopsis thaliana has a primary root elongation significantly higher than that of the wild-type control, and a root system aluminum content is significantly decreased; the ALR1-knockout mutant line has a primary root elongation significantly lower than that of the wild-type control, and the root system aluminum content is significantly higher than that of the wild-type control; and the mutant line with functional recovery has a primary root elongation similar to that of the wild-type control. This indicates that increasing the expression of ALR1 is beneficial to the improvement of plant aluminum resistance. The primer is capable of improving the plant aluminum resistance and promoting growth of a plant root system, thereby promoting absorption of nutrients and water by the plant, which is of great significance for improving a yield of crops. The plant seeds obtained by the primer, gene, protein or use of the present disclosure are used as aluminum-resistant plants, which can greatly simplify industrial production operations and open up channels for genetic breeding of aluminum-resistant plants. This provides a new production idea for genetic breeding of the aluminum-resistant plants, and reduces the screening process during the traditional breeding; in addition, the aluminum-resistant plants reduce chemicals and labor input for soil improvement, thereby greatly reducing production costs.

### BRIEF DESCRIPTION OF THE DRAWINGS

FIG. 1 shows a schematic diagram of a binary vector 35s-pCAMBIA1301 provided by the present disclosure;

FIG. 2 shows a schematic diagram of a transgenic vector pOEALR1 provided by the present disclosure;

FIG. 3 shows a comparison chart of an ALR1 gene expression level of a wild-type line and an ALR1-overexpressed transgenic line provided by the present disclosure;

FIG. 4 shows a comparison chart of aluminum resistances of the wild-type line, an ALR1 knockout mutant, a line with functional recovery, and an overexpression transgenic line provided by the present disclosure;

FIG. 5 shows a comparison chart of primary root relative elongations of the wild-type line, the ALR1 knockout mutant, the line with functional recovery, and the overexpression transgenic line provided by the present disclosure; and

FIG. 6 shows a comparison chart of aluminum contents of the wild-type line, the ALR1 knockout mutant, and the overexpression transgenic line provided by the present disclosure.

### DETAILED DESCRIPTION OF THE EMBODIMENTS

The present disclosure provides a primer of an ALR1 gene regulating plant aluminum resistance, where the primer includes an upstream primer with a nucleotide sequence shown in SEQ ID NO: 1 (5′-CGGATCCATGCGTGTTCATCGTTTTTGT-3′) and a downstream primer with a nucleotide sequence shown in SEQ ID NO: 2 (5′-CGTCGACCTAGACATCATCAAGCCAAGAG-3′). In the present disclosure, the primers are designed according to an ALR1 gene of an Arabidopsis thaliana aluminum ion receptor, and enzyme cleavage sites are designed at both ends to facilitate preparation of subsequent vectors. The gene has a nucleotide sequence shown in SEQ ID NO: 3 (ATGCGTGTTCATCGTTTTTGTGTGATCGTCATCTTCCTCACAGAGTTACTATGTTTCTTCTA TTCCTCGGAATCTCAGACCACCTCCAGGTGCCATCCACATGACCTCGAAGCCTTACGTGA CTTCATAGCACATCTCGAACCAAAACCAGATGGTTGGATCAATTCTTCTTCTTCTACAGAC TGCTGCAACTGGACCGGAATCACCTGCAATTCAAACAACACCGGAAGAGTTATTAGATTG GAGCTTGGGAACAAAAAGCTGTCGGGGAAGTTGTCTGAATCTCTCGGGAAGCTAGATGA GATTAGGGTTCTTAATCTCTCTCGAAACTTCATCAAAGATTCGATCCCTCTTTCGATTTTCA ACTTGAAGAATCTACAAACTCTTGATTTGAGCTCTAATGATCTCTCCGGCGGAATCCCAAC AAGTATAAATCTCCCAGCTCTGCAAAGTTTTGATCTTTCTTCAAATAAATTCAATGGGTCG CTTCCGTCTCATATCTGCCATAACTCTACTCAAATTAGGGTTGTGAAACTTGCGGTGAACT ACTTCGCCGGAAACTTCACTTCCGGGTTTGGGAAATGTGTCTTGCTTGAGCATCTCTGTC TTGGTATGAACGATCTTACTGGTAACATCCCTGAGGATTTGTTTCATCTCAAAAGATTGAA TCTTTTAGGGATTCAAGAGAATCGTCTCTCTGGTTCGTTGAGTCGTGAGATTAGGAATCTC TCAAGTCTTGTTCGTCTTGATGTTTCTTGGAATTTGTTTTCCGGTGAAATCCCTGATGTGTT CGACGAATTGCCTCAGTTAAAGTTTTTCTTAGGTCAGACCAATGGATTCATTGGAGGAATA CCTAAATCGTTGGCGAATTCACCGAGTTTGAATCTGCTTAACTTGAGGAACAATTCTTTAT CGGGTCGTTTGATGTTGAATTGTACGGCGATGATTGCTTTGAACTCTCTTGATTTAGGTAC CAATAGATTCAATGGGAGGTTACCTGAGAATCTACCGGATTGCAAGCGGTTAAAGAACGT TAACCTCGCGAGGAACACCTTCCATGGACAAGTACCAGAGAGTTTCAAGAACTTCGAGA GCTTATCTTACTTCTCGTTATCGAATTCGAGTTTGGCTAATATCTCTTCAGCGCTTGGGATA CTTCAGCATTGCAAGAACTTGACGACTTTGGTTCTTACATTGAATTTCCATGGAGAGGCTT TACCCGATGATTCAAGTCTTCATTTCGAGAAGCTTAAGGTGCTTGTAGTGGCGAATTGTAG GCTTACTGGTTCGATGCCGAGGTGGTTAAGCTCGAGTAATGAACTTCAGTTGTTGGATCTT TCTTGGAACCGTTTAACCGGCGCTATCCCGAGCTGGATTGGTGACTTCAAGGCTCTGTTC TACTTGGATTTATCTAACAACTCGTTTACAGGAGAGATCCCTAAGAGCTTAACTAAGTTAG AGAGTCTCACTAGCCGTAATATCTCAGTCAATGAGCCATCTCCTGATTTCCCGTTCTTTATG AAAAGAAACGAGAGCGCGAGAGCGTTGCAATACAATCAGATTTTCGGGTTCCCGCCAAC GATTGAGCTTGGTCATAACAATCTCTCTGGACCTATTTGGGAGGAGTTTGGTAATCTGAAG AAGCTTCATGTGTTTGATTTGAAATGGAATGCATTATCTGGATCAATACCTAGCTCGCTTTC TGGTATGACGAGCTTGGAAGCTCTTGATCTCTCTAATAACCGTCTTTCGGGTTCGATCCCG GTTTCTCTGCAACAGCTCTCGTTTCTGTCGAAGTTCAGTGTTGCTTATAACAATCTCTCGG GAGTAATACCTTCCGGTGGTCAGTTTCAGACGTTTCCAAACTCGAGCTTTGAGAGTAACC ATCTCTGCGGGGAACACAGATTCCCCTGTTCTGAAGGTACTGAGAGTGCATTGATCAAAC GGTCAAGAAGAAGCAGAGGAGGTGACATTGGAATGGCGATTGGGATAGCGTTTGGTTCG GTTTTTCTTTTGACTCTTCTCTCGTTGATTGTGTTGCGTGCTCGTAGACGGTCAGGAGAAG TTGATCCGGAGATAGAAGAATCCGAGAGCATGAATCGTAAAGAACTCGGAGAGATTGGA TCTAAGCTTGTGGTTTTGTTTCAGAGCAATGATAAAGAGCTCTCTTATGATGACCTTTTGG ACTCAACAAATAGTTTTGATCAAGCTAACATCATTGGCTGTGGCGGGTTTGGTATGGTTTA CAAAGCAACGTTACCAGACGGTAAGAAAGTTGCGATCAAGAAGTTATCCGGTGATTGCG GTCAAATCGAAAGAGAATTCGAAGCAGAAGTTGAAACACTCTCAAGAGCACAGCATCCA AATCTTGTTCTTCTCCGAGGATTCTGTTTCTACAAAAACGACCGGCTTTTAATCTACTCGT ATATGGAAAACGGAAGCTTAGACTATTGGCTACACGAGCGTAACGACGGTCCAGCGTTGT TGAAGTGGAAAACACGTCTTAGAATCGCTCAAGGTGCTGCAAAAGGGTTACTTTACTTGC ATGAAGGGTGTGATCCTCATATCTTACACCGCGATATTAAATCGAGTAATATTCTTCTCGAC GAGAATTTCAACTCTCATTTAGCGGATTTCGGACTCGCAAGGCTGATGAGTCCTTACGAG ACGCATGTAAGTACTGATTTGGTTGGAACTTTAGGTTACATTCCTCCGGAATACGGGCAAG CTTCGGTTGCTACTTACAAAGGCGATGTGTATAGTTTCGGAGTTGTGCTTCTCGAGCTTTT AACCGATAAAAGACCGGTGGATATGTGTAAACCGAAAGGGTGTAGGGATCTGATCTCGTG GGTCGTCAAGATGAAGCATGAGAGTCGAGCAAGCGAGGTTTTCGATCCGTTAATATACAG TAAAGAGAATGATAAAGAGATGTTTCGGGTTCTCGAGATTGCTTGTTTATGTTTAAGCGAA AACCCGAAACAGAGGCCAACGACTCAACAGTTAGTCTCTTGGCTTGATGATGTCTAG).

The primers can be used for artificial cloning of ALR1. There is no special limitation on a source of the ALR1 gene, and an artificial gene synthesis method or an amplification method well known in the art can be used. For example, the gene is preferably obtained by cloning; preferably, PCR amplification is conducted using a root system cDNA of Arabidopsis thaliana as a template with the primers above to obtain the ALR1 gene containing the enzyme cleavage sites at both ends. A reaction procedure of the PCR amplification includes preferably: initial denaturation at 94° C. for 2 min; denaturation at 98° C. for 10 sec, annealing at 57° C. for 30 sec, and extension at 68° C. for 3 min, conducting 30 cycles; and final extension at 68° C. for 5 min.

The plant includes preferably all types of plants, such as Arabidopsis thaliana. To illustrate regulation of the ALR1 gene, a model plant Arabidopsis thaliana is used as a material for an experiment, and an amplified gene is recorded as AtALR1 in examples.

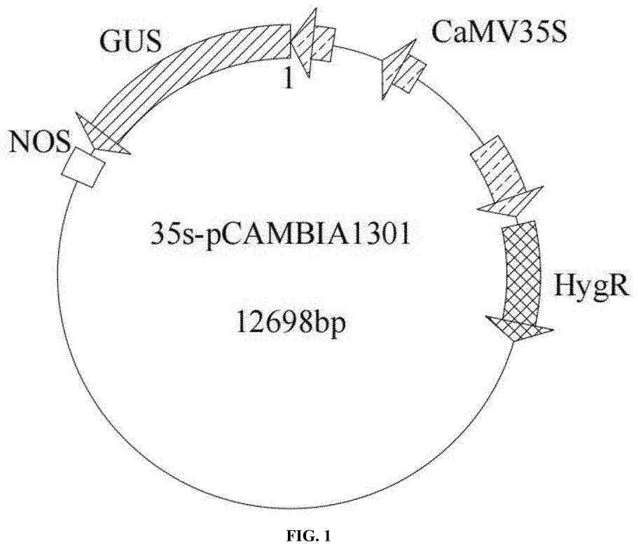

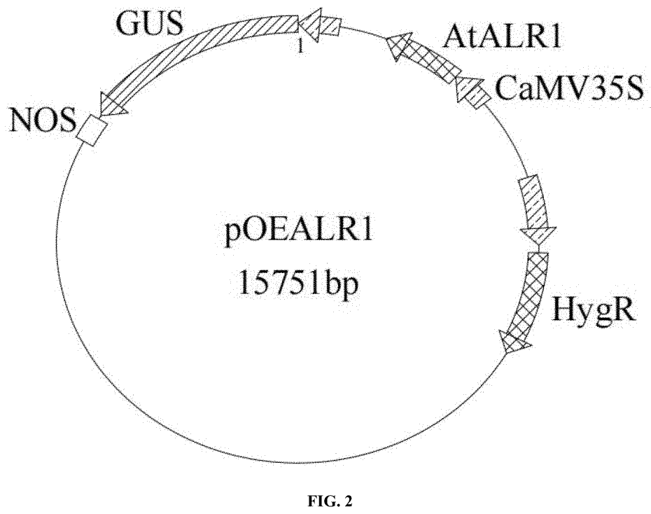

The present disclosure further provides a vector for overexpressing an ALR1 gene of an Arabidopsis thaliana aluminum ion receptor prepared based on the primer, where the vector uses 35s-pCAMBIA1301 as a vector backbone, and further includes an ALR1 gene of the Arabidopsis thaliana aluminum ion receptor; and the ALR1 gene has a nucleotide sequence shown in SEQ ID NO: 3. The vector for overexpressing the ALR1 gene of the aluminum ion receptor of Arabidopsis thaliana can overexpress the ALR1 gene of the aluminum ion receptor of the Arabidopsis thaliana. There is no special limitation on a construction method of the vector for overexpressing the ALR1 gene of the aluminum ion receptor of the Arabidopsis thaliana, and conventional vector construction methods well known to those skilled in the art can be used, such as an enzyme cleavage ligation method. In a specific example, preferably amplification is conducted using the upstream primer with a nucleotide sequence shown in SEQ ID NO: 1 and the downstream primer with a nucleotide sequence shown in SEQ ID NO: 2 to obtain an ALR1 encoding region sequence containing enzyme cleavage sites at both ends; the sequence is ligated to a vector pMD19T, and the ALR1 gene is excised from the vector pMD19T by double digestion with BamHI and SalI, and ligated to a binary vector pCAMBIA1301 (35s-pCAMBIA1301) containing a promoter CaMV35S; a constructed vector is named pOEALR1 (FIG. 2, a schematic diagram of the transgenic vector pOEALR1). The vector for overexpressing the ALR1 gene of the aluminum ion receptor of Arabidopsis thaliana is preferably transformed into a plant by an Agrobacterium-mediated method to overexpress the gene. The 35s-pCAMBIA1301 is a plant constitutive overexpression vector (FIG. 1, a schematic diagram of the binary vector 35s-pCAMBIA1301), which is a binary vector pCAMBIA1301 containing the promoter CaMV35S. A construction method of the 35s-pCAMBIA1301 preferably includes: inserting a cauliflower mosaic virus constitutive promoter CaMV35S into a multiple cloning site of the pCAMBIA1301 using enzyme cleavage sites SacI and Kpn1.

The present disclosure further provides use of an ALR1 gene or an ALR1 protein of an Arabidopsis thaliana aluminum ion receptor or the primer in regulating plant aluminum resistance, where the ALR1 gene has a nucleotide sequence shown in SEQ ID NO: 3, and the ALR1 protein has an amino acid sequence shown in SEQ ID NO: 4 (MRVHRFCVIVIFLTELLCFFYSSESQTTSRCHPHDLEALRDFIAHLEPKPDGWINSSSSTDCCN WTGITCNSNNTGRVIRLELGNKKLSGKLSESLGKLDEIRVLNLSRNFIKDSIPLSIFNLKNLQT LDLSSNDLSGGIPTSINLPALQSFDLSSNKFNGSLPSHICHNSTQIRVVKLAVNYFAGNFTSGFG KCVLLEHLCLGMNDLTGNIPEDLFHLKRLNLLGIQENRLSGSLSREIRNLSSLVRLDVSWNLF SGEIPDVFDELPQLKFFLGQTNGFIGGIPKSLANSPSLNLLNLRNNSLSGRLMLNCTAMIALNS LDLGTNRFNGRLPENLPDCKRLKNVNLARNTFHGQVPESFKNFESLSYFSLSNSSLANIS SAL GILQHCKNLTTLVLTLNFHGEALPDDSSLHFEKLKVLVVANCRLTGSMPRWLSSSNELQLLDL SWNRLTGAIPSWIGDFKALFYLDLSNNSFTGEIPKSLTKLESLTSRNISVNEPSPDFPFFMKRNE SARALQYNQIFGFPPTIELGHNNLSGPIWEEFGNLKKLHVFDLKWNALSGSIPSSLSGMTSLE ALDLSNNRLSGSIPVSLQQLSFLSKFSVAYNNLSGVIPSGGQFQTFPNSSFESNHLCGEHRFPC SEGTESALIKRSRRSRGGDIGMAIGIAFGSVFLLTLLSLIVLRARRRSGEVDPEIEESESMNRKE LGEIGSKLVVLFQSNDKELSYDDLLDSTNSFDQANIIGCGGFGMVYKATLPDGKKVAIKKLS GDCGQIEREFEAEVETLSRAQHPNLVLLRGFCFYKNDRLLIYSYMENGSLDYWLHERNDGP ALLKWKTRLRIAQGAAKGLLYLHEGCDPHILHRDIKSSNILLDENFNSHLADFGLARLMSPY ETHVSTDLVGTLGYIPPEYGQASVATYKGDVYSFGVVLLELLTDKRPVDMCKPKGCRDLISW VVKMKHESRASEVFDPLIYSKENDKEMFRVLEIACLCLSENPKQRPTTQQLVSWLDDV). The ALR1 is located in an encoding region 1 to 3027 of a full-length cDNA of the ALR1, with a nucleotide sequence of 3027 bp; and the protein is a sequence including 1008 amino acids. The plant aluminum resistance is evaluated preferably by using aluminum treatment to detect an elongation of a primary root of the plant. The plant includes preferably all types of plants, such as Arabidopsis thaliana. To illustrate regulation of the ALR1 gene, a model plant Arabidopsis thaliana is used as a material for an experiment.

The present disclosure further provides use of an ALR1 gene or an ALR1 protein for overexpressing an Arabidopsis thaliana aluminum ion receptor or the vector in improving plant aluminum resistance, where the ALR1 gene has a nucleotide sequence shown in SEQ ID NO: 3, and the ALR1 protein has an amino acid sequence shown in SEQ ID NO: 4. The overexpression method specifically includes preferably the following steps: cloning the ALR1 gene into a plant constitutive overexpression vector to obtain a recombinant expression vector; conducting Agrobacterium-mediated transformation on the recombinant expression vector into a plant. The plant constitutive overexpression vector is preferably 35s-pCAMBIA1301, and a construction method of the 35s-pCAMBIA1301 is preferably as described above. There is no special limitation on a transformation method, and conventional operation methods well known to those skilled in the art can be used. After the transformation, culturing, screening and harvesting transgenic seeds are preferably conducted. There is no special limitation on a method for the culturing, screening and harvesting, and conventional operation methods well known to those skilled in the art can be used. The plant includes preferably all types of plants, such as Arabidopsis thaliana. To illustrate overexpression method of the ALR1 gene, a model plant Arabidopsis thaliana is used as a material for an experiment.

The present disclosure further provides use of an ALR1 gene or an ALR1 protein of an Arabidopsis thaliana aluminum ion receptor or the primer in regulating a root system elongation of an aluminum-stressed plant, where the ALR1 gene has a nucleotide sequence shown in SEQ ID NO: 3, and the ALR1 protein has an amino acid sequence shown in SEQ ID NO: 4. The regulation includes preferably promoting the elongation of plant root systems through overexpression of ALR1 gene or inhibiting the elongation of plant root systems through knockout or silencing of the ALR1 gene. There is no specific limitation on a method of the gene knockout or silencing, and gene knockout or silencing methods well-known in the art can be used. The plant includes preferably all types of plants, such as Arabidopsis thaliana. To illustrate regulation of the ALR1 gene, a model plant Arabidopsis thaliana is used as a material for an experiment.

The present disclosure further provides use of an ALR1 gene or an ALR1 protein for overexpressing an Arabidopsis thaliana aluminum ion receptor or the vector in regulating a root system elongation of an aluminum-stressed plant, where the ALR1 gene has a nucleotide sequence shown in SEQ ID NO: 3, and the ALR1 protein has an amino acid sequence shown in SEQ ID NO: 4. The plant includes preferably all types of plants, such as Arabidopsis thaliana. To illustrate overexpression method of the ALR1 gene, a model plant Arabidopsis thaliana is used as a material for an experiment. In an example, overexpression of the ALR1 gene increases a root system elongation of Arabidopsis thaliana under aluminum stress; a relative elongation of Arabidopsis thaliana root system is significantly higher than that of the wild-type and ALR1-knockout mutant materials of Arabidopsis thaliana. The root system is preferably the primary root. In an example, overexpression of the ALR1 gene increases a root system elongation of Arabidopsis thaliana under aluminum stress; a relative elongation of Arabidopsis thaliana primary root is significantly higher than that of the wild-type and ALR1-knockout mutant materials of Arabidopsis thaliana.

In the present disclosure, the overexpression method is preferably the same as that for use of an ALR1 gene or an ALR1 protein for overexpressing an Arabidopsis thaliana aluminum ion receptor or the vector in improving plant aluminum resistance. A method for detecting the elongation of primary root includes preferably the following steps: disinfecting plant seeds with 75% alcohol, and washing with sterilized water for 3 to 5 times; dibbling the seeds on a ½ MS solid medium plate, and placing the plate in a refrigerator at 4° C. for 2 d to 3 d; and placing the plate in a light incubator (illumination for 16 h/darkness for 8 h) for 7 d to 10 d. A length of the seedling primary root of each material is measured by a ruler; a relative elongation of the corresponding material under control conditions (without aluminum treatment) is calculated, namely: root length under control conditions/average root length under control conditions*100; and a relative elongation of the corresponding material under aluminum treatment is calculated, namely: root length under aluminum treatment/average root length under control conditions*100.

The present disclosure further provides use of an ALR1 gene or an ALR1 protein of an Arabidopsis thaliana aluminum ion receptor or the primer in regulating a root system aluminum content of an aluminum-stressed plant, where the ALR1 gene has a nucleotide sequence shown in SEQ ID NO: 3, and the ALR1 protein has an amino acid sequence shown in SEQ ID NO: 4. The regulation includes preferably reducing the aluminum content of plant root systems through overexpression of ALR1 gene or improving the aluminum content of plant root systems through knockout or silencing of the ALR1 gene. The plant includes preferably all types of plants, such as Arabidopsis thaliana. To illustrate regulation of the ALR1 gene, a model plant Arabidopsis thaliana is used as a material for an experiment.

The present disclosure further provides use of an ALR1 gene or an ALR1 protein for overexpressing an Arabidopsis thaliana aluminum ion receptor or the vector in regulating a root system aluminum content of an aluminum-stressed plant, where the ALR1 gene has a nucleotide sequence shown in SEQ ID NO: 3, and the ALR1 protein has an amino acid sequence shown in SEQ ID NO: 4. The plant includes preferably all types of plants, such as Arabidopsis thaliana. To illustrate overexpression method of the ALR1 gene, a model plant Arabidopsis thaliana is used as a material for an experiment. In an example, overexpression of the ALR1 gene reduce an aluminum content of Arabidopsis thaliana under aluminum stress; the aluminum content of Arabidopsis thaliana root system is significantly lower than that of the wild-type and ALR1-knockout mutant materials of Arabidopsis thaliana.

In the present disclosure, the overexpression method is preferably the same as that for use of an ALR1 gene or an ALR1 protein for overexpressing an Arabidopsis thaliana aluminum ion receptor or the vector in improving plant aluminum resistance. A method for detecting the aluminum content in root system includes preferably the following steps: treating seedlings with a 0.5 mM calcium chloride solution and a 50 μM aluminum chloride solution for 24 h; rinsing seedling roots three times with ultrapure water to remove the aluminum solution on a surface of the root, and removing the ultrapure water with a filter paper; cutting off a primary root of the seedlings with a clean blade, and combining the roots of a same line for weighing; conducting digestion and cracking on the roots with a mixed solution of nitric acid and perchloric acid (at 4:1 by volume); filtering a fully-lysed sample through the filter paper and collecting in a clean tube for testing; and determining an aluminum content in an extract by inductively coupled plasma-atomic emission spectrometry (ICP-AES).

The use of an ALR1 gene or an ALR1 protein of an aluminum ion receptor in regulating plant aluminum resistance according to the present disclosure will be further described in detail below with reference to specific examples. The technical solutions of the present disclosure include, but are not limited to, the following examples.

### Example 1

Cloning of ALR1 Gene

Surface-sterilized Arabidopsis thaliana seeds were planted in a ½ MS solid medium, vernalized at 4° C. in the dark for 2 d to 3 d, and transferred to illumination for 6 d; the roots of seedlings were collected for RNA extraction, and reverse transcription was conducted by a kit to synthesize a cDNA as a template for subsequent gene cloning.

According to published whole genome sequencing results of Arabidopsis thaliana, upstream and downstream primers were designed separately as follows:

upstream primer:
(SEQ ID NO: 5)
5′-ATGCTGTTCATCGTTTTTGT-3′;
and
downstream primer:
(SEQ ID NO: 6)
5′-CTAGACATCATCAAGCCAAGAG-3′.

PCR amplification was conducted using a KOD FX enzyme (TOYOBO). A reaction procedure of the PCR amplification included: initial denaturation at 94° C. for 2 min; denaturation at 98° C. for 10 sec, annealing at 57° C. for 30 sec, and extension at 68° C. for 3 min, conducting 30 cycles; and final extension at 68° C. for 5 min. A system of the PCR amplification included:

| | | |
|---|---|---|
| 2 × PCR buffer for KOD FX | 25 | μl |
| 2 mM dNTPs | 10 | μl |
| 10 pmol 5′ primer | 1.5 | μl |
| 10 pmol 3′ primer | 1.5 | μl |
| cDNA | 2 | μl |
| KOD FX enzyme | 1 | μl |
| ddH₂O | 9 | μl. |

A PCR amplification product was sent for sequencing to obtain a CDS sequence of AtALR1 (SEQ ID NO: 3).

### Example 2

Construction of Constitutive Overexpression Transgenic Vector

A cauliflower mosaic virus constitutive promoter CaMV35S was inserted into a multiple cloning site using a method of double digestion and ligation of DNA fragments through enzyme cleavage sites SacI and Kpn1, such that a promoter CaMV35S was successfully ligated to a pCAMBIA1301 vector, and a vector 35s-pCAMBIA1301 capable of constructing a constitutive overexpression transgenic material was obtained through transformation (FIG. 1).

Amplification was conducted using primers ALR1-F: 5′-CGGATCCATGCGTGTTCATCGTTTTTGT-3′ (SEQ ID NO: 1) and ALR1-R: 5′-CGTCGACCTAGACATCATCAAGCCAAGAG-3′ (SEQ ID NO: 2), with the cDNA sequence obtained in Example 1 as a template, and referring to the PCR amplification reaction program in Example 1, to obtain an encoding region sequence of an aluminum ion receptor gene AtALR1 of Arabidopsis thaliana containing enzyme cleavage sites at both ends. According to instructions of a pMD19T vector produced by Takara Company, the encoding region sequence of an aluminum ion receptor gene AtALR1 of Arabidopsis thaliana was ligated to the pMD19T; the encoding region sequence of an aluminum ion receptor gene AtALR1 of Arabidopsis thaliana was excised from the pMD19T vector and ligated to the promoter CaMV35S on the constitutive overexpression vector 35s-pCAMBIA1301 using a method of BamHI and SalI double digestion and ligation, to obtain a transgenic vector pOEALR1 (binary transgenic vector pOEALR1 plasmid) that promotes the Arabidopsis thaliana gene AtALR1 by the promoter CaMV35S (FIG. 2).

### Example 3

Transformation of Arabidopsis thaliana

0.5 μg of the binary transgenic vector pOEALR1 plasmid prepared in Example 2 was transferred into competent cells of an Agrobacterium tumefaciens strain GV3101; ice bath for 5 min, liquid nitrogen treatment for 5 min, water bath at 37° C. for 5 min, and ice bath for 5 min were conducted in sequence; and the cells were added to a non-antigenic LB medium and activated in a shaker at 28° C. for 1 h to obtain an Agrobacterium tumefaciens strain containing a binary plasmid vector. The Arabidopsis thaliana was transformed using the GV3101 strain containing a binary plasmid vector, and specific steps were as follows:

The Agrobacterium tumefaciens strain containing a binary plasmid vector was cultured in an LB medium containing 50 mg/L kanamycin (Kan) and 50 mg/L rifampicin (Rif) at 28° C. with shaking overnight to an OD600 absorbance value of 1.0, bacterial cells were collected after centrifugation at 4,000 rpm for 15 min, and then resuspended in a ½ MS medium containing 50 g/L sucrose. The wild-type (Col-0) Arabidopsis thaliana that had been bolted and partially completed flowering was selected as a transgenic material, mature pods were removed, and flowers and buds were retained; by a vacuum transformation method, aerial parts of the Arabidopsis thaliana were immersed in the bacterial solution prepared above, vacuumized for 5 min, cultured for 24 h in the dark at 23° C., screened on the ½MS medium containing 50 mg/L hygromycin for 1 week to obtain resistant seedlings; and the resistant seedlings were transplanted into soil for culture, and transgenic first-generation (T1 generation) seeds were harvested. The T1 generation seeds were screened for another generation on the ½MS medium containing 50 mg/L hygromycin, to obtain a homozygous transgenic T2 generation material (ALR1 ox 1).

### Example 4

Molecular Detection of Target Gene Expression

The wild-type and overexpression transgenic plants were sampled to extract RNA from young whole plantlets. After reverse transcription, real-time quantitative PCR detection was conducted using a SYBR Green Realtime PCR Master Mix of TOYOBO Company, where a Actin2 gene was used as an internal reference; a detection system and primers used were as follows:

The primers used for real-time quantitative PCR were:

qALR1-F:
(SEQ ID NO: 7)
5′-AGCGAGGTTTTCGATCCGTT-3′
qALR1-R:
(SEQ ID NO: 8)
5′-CTGTTGAGTCGTTGGCCTCT-3′
qActin2-F:
(SEQ ID NO: 9)
5′-GGTAACATTGTGCTCAGTGGTGG-3′
qActin2-R:
(SEQ ID NO: 10)
5′-AACGACCTTAATCTTCATGCTGC-3′.

A reaction procedure of real-time quantitative PCR was as follows:

initial denaturation: 95° C., 1 min; PCR cycles: 95° C., 15 sec; 60° C., 15 sec; 72° C., 45 sec (40 cycles).

A reaction system of real-time quantitative PCR included:

| | | |
|---|---|---|
| SYBR Green Realtime PCR Master Mix | 5 | μl |
| qALR1-F (10 μM) | 0.1 | μl |
| qALR1-R (10 μM) | 0.1 | μl |
| Ultrapure water | 4.6 | μL |
| Reverse transcription product (1000 ng) | 0.2 | μl |

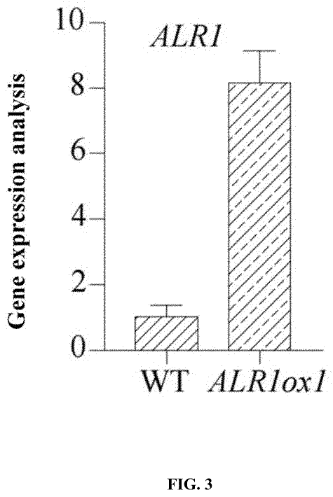

After detection, it is found that, as shown in FIG. 3 (FIG. 3 shows a comparison chart of an ALR1 gene expression level of a wild-type line and an ALR1-overexpressed transgenic line); the ALR1 gene in non-transgenic lines has an expression level of 1.00±0.36, while the ALR1 gene in overexpression transgenic plants has an expression level of 8.15±0.97. Compared with the non-transgenic lines, the ALR1 gene is 15 to 20 times higher in the overexpression transgenic plants.

### Example 5

Detection of Aluminum Resistance in Seeds

Evaluation of plant aluminum resistance was conducted by detecting a relative elongation of Arabidopsis thaliana primary root by aluminum treatment. The wild-type plants and Arabidopsis thaliana ALR1-knockout mutant materials (alr1) purchased from the Arabidopsis thaliana Biological Resource Center (ABRC) were selected; a mutant material with functional recovery constructed by re-transferring the ALR1 gene into the alr1 mutant, and seeds of the overexpression transgenic material obtained in Example 3 were surface-sterilized with 75% alcohol, and then washed with sterile water 3 to 5 times; the seeds were dibbled on ½ MS solid medium plates with (aluminum treatment) or without 1 mM aluminum chloride (without aluminum treatment as a control), and the plates were placed in a 4° C. refrigerator for 2 d to 3 d; and the plates were placed in a light incubator (illumination for 16 h/darkness for 8 h) for 7 d to 10 d of growth. A length of the seedling primary root of each material is measured by a ruler; a relative elongation of the corresponding material under control conditions (without aluminum treatment) is calculated, namely: root length under control conditions/average root length under control conditions*100; and a relative elongation of the corresponding material under aluminum treatment is calculated, namely: root length under aluminum treatment/average root length under control conditions*100.

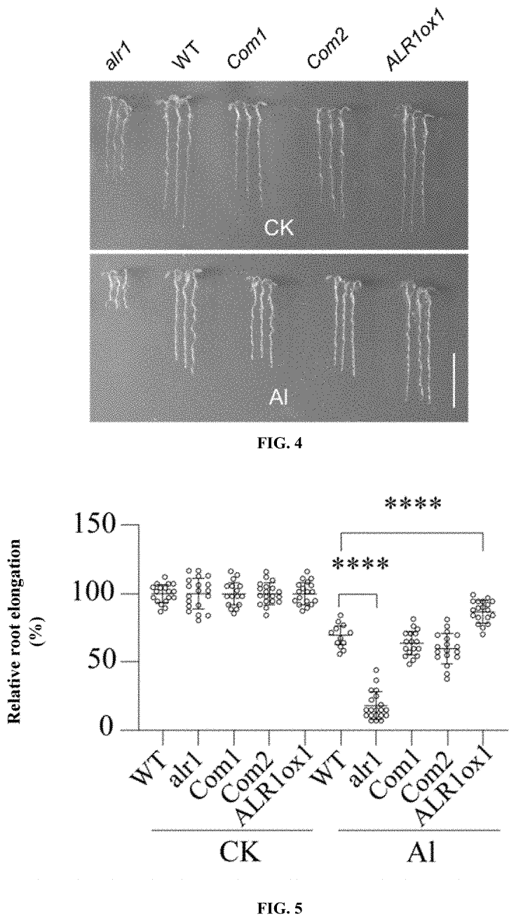

The results are shown in FIG. 4 (a comparison chart of aluminum resistances of the wild-type line, an ALR1 knockout mutant, and an overexpression transgenic line; where a length of the scale is 1 cm), FIG. 5 (a comparison chart of primary root relative elongations of the wild-type line, the ALR1 knockout mutant, the line with functional recovery, and the overexpression transgenic line), and Table 1. Under no aluminum stress, the root lengths of the overexpression transgenic Arabidopsis thaliana (ALR1 ox 1), ALR1-knockout mutant material (alr1) and material with functional recovery (Com1 and Com2) were not significantly different from that of the wild-type control; while under aluminum stress, the relative elongation of primary root of overexpression transgenic Arabidopsis thaliana (ALR1 ox 1) is significantly higher than that of the wild-type control, and the relative elongation of primary root of the ALR1-knockout mutant line is significantly lower than that of the wild-type control.

TABLE 1
Relative elongation of primary root (%)

| | CK | | | | | Al | | | | |
|---|---|---|---|---|---|---|---|---|---|---|
| No. | WT | alr1 | Com1 | Com2 | ALR1ox1 | WT | alr1 | Com1 | Com2 | ALR1ox1 |
| 1 | 104.59 | 98.54 | 100.00 | 98.27 | 101.45 | 66.33 | 10.95 | 64.86 | 57.80 | 84.54 |
| 2 | 96.94 | 105.84 | 97.30 | 95.38 | 106.28 | 68.88 | 14.60 | 59.46 | 54.91 | 77.29 |
| 3 | 96.94 | 116.79 | 102.70 | 104.05 | 94.20 | 84.18 | 7.30 | 62.16 | 46.24 | 82.13 |
| 4 | 99.49 | 102.19 | 105.41 | 92.49 | 89.37 | 76.53 | 25.55 | 70.27 | 75.14 | 94.20 |
| 5 | 112.24 | 98.54 | 91.89 | 89.60 | 101.45 | 56.12 | 29.20 | 75.68 | 40.46 | 94.20 |
| 6 | 91.84 | 94.89 | 97.30 | 109.83 | 108.70 | 63.78 | 7.30 | 59.46 | 80.92 | 70.05 |
| 7 | 94.39 | 102.19 | 110.81 | 115.61 | 111.11 | 71.43 | 10.95 | 54.05 | 49.13 | 86.96 |
| 8 | 107.14 | 105.84 | 113.51 | 112.72 | 94.20 | 68.88 | 18.25 | 56.76 | 57.80 | 94.20 |
| 9 | 102.04 | 83.94 | 97.30 | 98.27 | 91.79 | 73.98 | 14.60 | 81.08 | 60.69 | 99.03 |
| 10 | 107.14 | 113.14 | 105.41 | 104.05 | 115.94 | 76.53 | 10.95 | 59.46 | 60.69 | 84.54 |
| 11 | 104.59 | 91.24 | 102.70 | 106.94 | 99.03 | 68.88 | 14.60 | 48.65 | 63.58 | 82.13 |
| 12 | 104.59 | 91.24 | 89.19 | 101.16 | 106.28 | 63.78 | 21.90 | 72.97 | 69.36 | 89.37 |
| 13 | 107.14 | 80.29 | 91.89 | 95.38 | 111.11 | 61.22 | 7.30 | 70.27 | 72.25 | 74.88 |
| 14 | 102.04 | 113.14 | 105.41 | 92.49 | 91.79 | 68.88 | 10.95 | 64.86 | 37.57 | 77.29 |
| 15 | 96.94 | 87.59 | 116.22 | 104.05 | 94.20 | 71.43 | 43.80 | 51.35 | 66.47 | 94.20 |
| 16 | 94.39 | 105.84 | 86.49 | 98.27 | 86.96 | 58.67 | 36.50 | 54.05 | 54.91 | 91.79 |
| 17 | 86.73 | 109.49 | 89.19 | 101.16 | 91.79 | 68.88 | 18.25 | 64.86 | 69.36 | 84.54 |
| 18 | 89.29 | 83.94 | 97.30 | 95.38 | 101.45 | 79.08 | 29.20 | 62.16 | 54.91 | 89.37 |
| 19 | 102.04 | 116.79 | 100.00 | 83.82 | 103.86 | 71.43 | 10.95 | 70.27 | 60.69 | 79.71 |
| 20 | 99.49 | 98.54 | 97.30 | 98.27 | 96.62 | 68.88 | 14.60 | 64.86 | 57.80 | 96.62 |

Detection of Aluminum Content in Root System

Seedlings growing for 7 d from the wild-type plant, the Arabidopsis thaliana ALR1-knockout mutant material (alr1) purchased from the Arabidopsis thaliana Biological Resource Center (ABRC), and the overexpression transgenic material obtained in Example 3 were treated by 0.5 mM calcium chloride solution and a 50 μM aluminum chloride solution (Al) for 24 h; seedling roots were rinsed three times with ultrapure water to remove the aluminum solution on a surface of the root, and the ultrapure water was removed with a filter paper; a primary root of the seedlings was cut off with a clean blade, and the roots of a same line were combined for weighing; digestion and cracking were conducted on the roots with a mixed solution of nitric acid and perchloric acid (at 4:1 by volume); a fully-lysed sample was filtered through the filter paper and collected in a clean tube for testing; and an aluminum content in an extract was determined by ICP-AES.

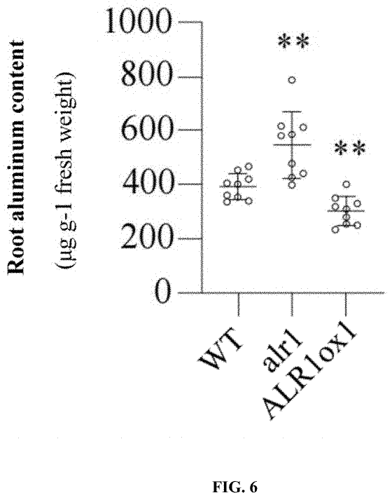

The results are shown in FIG. 6 (a comparison chart of aluminum contents of the wild-type line, the ALR1 knockout mutant, and the overexpression transgenic line) and Table 2. Under aluminum stress, the root system aluminum content of overexpression transgenic Arabidopsis thaliana (ALR1 ox 1) is significantly lower than that of the wild-type control, while the root system aluminum content of ALR1-knockout mutant line is significantly higher than that of the wild-type control.

TABLE 2
Aluminum content of root system (μg/g)

| No. | WT | ALR1 | ALR1ox1 |
|---|---|---|---|
| 1 | 403.2573 | 789.3843 | 350.5266 |
| 2 | 356.3158 | 587.0902 | 400.3358 |
| 3 | 340.1515 | 478.5032 | 280.5766 |
| 4 | 360.6925 | 428.1437 | 320.1371 |
| 5 | 405.4054 | 399.7126 | 312.8168 |
| 6 | 467.3469 | 617.551 | 250.6913 |
| 7 | 452.0629 | 440 | 330.581 |
| 8 | 338.4137 | 610.2334 | 236.6282 |
| 9 | 420.6221 | 580.6346 | 258.4818 |

It can be seen that the aluminum resistance of overexpression transgenic Arabidopsis thaliana (ALR1 ox 1) is significantly higher than that of the wild-type control, while the aluminum resistance of the ALR1-knockout mutant line is significantly lower than that of the wild-type control, indicating that the gene ALR1 has involved in the regulation of plant aluminum resistance.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

1-10. (canceled)

11. A method for regulating plant aluminum resistance by an ALR1 gene or an ALR1 protein of an Arabidopsis thaliana aluminum ion receptor, wherein the ALR1 gene has a nucleotide sequence shown in SEQ ID NO: 3, and the ALR1 protein has an amino acid sequence shown in SEQ ID NO: 4.

12. A method for regulating a root system elongation of an aluminum-stressed plant by an ALR1 gene or an ALR1 protein of an Arabidopsis thaliana aluminum ion receptor, wherein the ALR1 gene has a nucleotide sequence shown in SEQ ID NO: 3, and the ALR1 protein has an amino acid sequence shown in SEQ ID NO: 4.

13. A method for regulating a root system aluminum content of an aluminum-stressed plant by an ALR1 gene or an ALR1 protein of an Arabidopsis thaliana aluminum ion receptor, wherein the ALR1 gene has a nucleotide sequence shown in SEQ ID NO: 3, and the ALR1 protein has an amino acid sequence shown in SEQ ID NO: 4.

14. The method according to claim 11, wherein the plant comprises Arabidopsis thaliana.

15. The method according to claim 12, wherein the plant comprises Arabidopsis thaliana.

16. The method according to claim 13, wherein the plant comprises Arabidopsis thaliana.


SEQUENCE LISTING (10 sequences)

SEQ ID NO: 1 - DNA, 28 bp, Artificial Sequence
  Upstream primer ALR1-F
cggatccatgcgtgttcatcgtttttgt

SEQ ID NO: 2 - DNA, 29 bp, Artificial Sequence
  Downstream primer ALR1-R
cgtcgacctagacatcatcaagccaagag

SEQ ID NO: 3 - DNA, 3027 bp, Artificial Sequence
  Nucleotide sequence of ALR1
atgcgtgttcatcgtttttgtgtgatcgtcatcttcctcacagagttactatgtttcttctattcctcggaatctcagaccacctccaggtgccatccacatgacctcgaagccttacgtgacttcatagcacatctcgaaccaaaaccagatggttggatcaattcttcttcttctacagactgctgcaactggaccggaatcacctgcaattcaaacaacaccggaagagttattagattggagcttgggaacaaaaagctgtcggggaagttgtctgaatctctcgggaagctagatgagattagggttcttaatctctctcgaaacttcatcaaagattcgatccctctttcgattttcaacttgaagaatctacaaactcttgatttgagctctaatgatctctccggcggaatcccaacaagtataaatctcccagctctgcaaagttttgatctttcttcaaataaattcaatgggtcgcttccgtctcatatctgccataactctactcaaattagggttgtgaaacttgcggtgaactacttcgccggaaacttcacttccgggtttgggaaatgtgtcttgcttgagcatctctgtcttggtatgaacgatcttactggtaacatccctgaggatttgtttcatctcaaaagattgaatcttttagggattcaagagaatcgtctctctggttcgttgagtcgtgagattaggaatctctcaagtcttgttcgtcttgatgtttcttggaatttgttttccggtgaaatccctgatgtgttcgacgaattgcctcagttaaagtttttcttaggtcagaccaatggattcattggaggaatacctaaatcgttggcgaattcaccgagtttgaatctgcttaacttgaggaacaattctttatcgggtcgtttgatgttgaattgtacggcgatgattgctttgaactctcttgatttaggtaccaatagattcaatgggaggttacctgagaatctaccggattgcaagcggttaaagaacgttaacctcgcgaggaacaccttccatggacaagtaccagagagtttcaagaacttcgagagcttatcttacttctcgttatcgaattcgagtttggctaatatctcttcagcgcttgggatacttcagcattgcaagaacttgacgactttggttcttacattgaatttccatggagaggctttacccgatgattcaagtcttcatttcgagaagcttaaggtgcttgtagtggcgaattgtaggcttactggttcgatgccgaggtggttaagctcgagtaatgaacttcagttgttggatctttcttggaaccgtttaaccggcgctatcccgagctggattggtgacttcaaggctctgttctacttggatttatctaacaactcgtttacaggagagatccctaagagcttaactaagttagagagtctcactagccgtaatatctcagtcaatgagccatctcctgatttcccgttctttatgaaaagaaacgagagcgcgagagcgttgcaatacaatcagattttcgggttcccgccaacgattgagcttggtcataacaatctctctggacctatttgggaggagtttggtaatctgaagaagcttcatgtgtttgatttgaaatggaatgcattatctggatcaatacctagctcgctttctggtatgacgagcttggaagctcttgatctctctaataaccgtctttcgggttcgatcccggtttctctgcaacagctctcgtttctgtcgaagttcagtgttgcttataacaatctctcgggagtaataccttccggtggtcagtttcagacgtttccaaactcgagctttgagagtaaccatctctgcggggaacacagattcccctgttctgaaggtactgagagtgcattgatcaaacggtcaagaagaagcagaggaggtgacattggaatggcgattgggatagcgtttggttcggtttttcttttgactcttctctcgttgattgtgttgcgtgctcgtagacggtcaggagaagttgatccggagatagaagaatccgagagcatgaatcgtaaagaactcggagagattggatctaagcttgtggttttgtttcagagcaatgataaagagctctcttatgatgaccttttggactcaacaaatagttttgatcaagctaacatcattggctgtggcgggtttggtatggtttacaaagcaacgttaccagacggtaagaaagttgcgatcaagaagttatccggtgattgcggtcaaatcgaaagagaattcgaagcagaagttgaaacactctcaagagcacagcatccaaatcttgttcttctccgaggattctgtttctacaaaaacgaccggcttttaatctactcgtatatggaaaacggaagcttagactattggctacacgagcgtaacgacggtccagcgttgttgaagtggaaaacacgtcttagaatcgctcaaggtgctgcaaaagggttactttacttgcatgaagggtgtgatcctcatatcttacaccgcgatattaaatcgagtaatattcttctcgacgagaatttcaactctcatttagcggatttcggactcgcaaggctgatgagtccttacgagacgcatgtaagtactgatttggttggaactttaggttacattcctccggaatacgggcaagcttcggttgctacttacaaaggcgatgtgtatagtttcggagttgtgcttctcgagcttttaaccgataaaagaccggtggatatgtgtaaaccgaaagggtgtagggatctgatctcgtgggtcgtcaagatgaagcatgagagtcgagcaagcgaggttttcgatccgttaatatacagtaaagagaatgataaagagatgtttcgggttctcgagattgcttgtttatgtttaagcgaaaacccgaaacagaggccaacgactcaacagttagtctcttggcttgatgatgtctag

SEQ ID NO: 4 - protein, 1008 aa, Artificial Sequence
  Amino acid sequence of ALR1
MRVHRFCVIVIFLTELLCFFYSSESQTTSRCHPHDLEALRDFIAHLEPKPDGWINSSSSTDCCNWTGITCNSNNTGRVIRLELGNKKLSGKLSESLGKLDEIRVLNLSRNFIKDSIPLSIFNLKNLQTLDLSSNDLSGGIPTSINLPALQSFDLSSNKFNGSLPSHICHNSTQIRVVKLAVNYFAGNFTSGFGKCVLLEHLCLGMNDLTGNIPEDLFHLKRLNLLGIQENRLSGSLSREIRNLSSLVRLDVSWNLFSGEIPDVFDELPQLKFFLGQTNGFIGGIPKSLANSPSLNLLNLRNNSLSGRLMLNCTAMIALNSLDLGTNRFNGRLPENLPDCKRLKNVNLARNTFHGQVPESFKNFESLSYFSLSNSSLANISSALGILQHCKNLTTLVLTLNFHGEALPDDSSLHFEKLKVLVVANCRLTGSMPRWLSSSNELQLLDLSWNRLTGAIPSWIGDFKALFYLDLSNNSFTGEIPKSLTKLESLTSRNISVNEPSPDFPFFMKRNESARALQYNQIFGFPPTIELGHNNLSGPIWEEFGNLKKLHVFDLKWNALSGSIPSSLSGMTSLEALDLSNNRLSGSIPVSLQQLSFLSKFSVAYNNLSGVIPSGGQFQTFPNSSFESNHLCGEHRFPCSEGTESALIKRSRRSRGGDIGMAIGIAFGSVFLLTLLSLIVLRARRRSGEVDPEIEESESMNRKELGEIGSKLVVLFQSNDKELSYDDLLDSTNSFDQANIIGCGGFGMVYKATLPDGKKVAIKKLSGDCGQIEREFEAEVETLSRAQHPNLVLLRGFCFYKNDRLLIYSYMENGSLDYWLHERNDGPALLKWKTRLRIAQGAAKGLLYLHEGCDPHILHRDIKSSNILLDENFNSHLADFGLARLMSPYETHVSTDLVGTLGYIPPEYGQASVATYKGDVYSFGVVLLELLTDKRPVDMCKPKGCRDLISWVVKMKHESRASEVFDPLIYSKENDKEMFRVLEIACLCLSENPKQRPTTQQLVSWLDDV

SEQ ID NO: 5 - DNA, 20 bp, Artificial Sequence
  Upstream primer of Arabidopsis thaliana
atgctgttcatcgtttttgt

SEQ ID NO: 6 - DNA, 22 bp, Artificial Sequence
  Downstream primer of Arabidopsis thaliana
ctagacatcatcaagccaagag

SEQ ID NO: 7 - DNA, 20 bp, Artificial Sequence
  Primer qALR1-F
agcgaggttttcgatccgtt

SEQ ID NO: 8 - DNA, 20 bp, Artificial Sequence
  Primer qALR1-R
ctgttgagtcgttggcctct

SEQ ID NO: 9 - DNA, 23 bp, Artificial Sequence
  Primer qActin2-F
ggtaacattgtgctcagtggtgg

SEQ ID NO: 10 - DNA, 23 bp, Artificial Sequence
  Primer qActin2-R
aacgaccttaatcttcatgctgc